(19) United States
(12) Patent Application Publication
(10) Pub. No.: US 2023/0397915 A1
(43) Pub. Date: Dec. 14, 2023

(54) Surgical Staple For Clamping Biological Tissue

(71) Applicant: Peters Surgical, Boulogne-Billancourt (FR)

(72) Inventor: Denis Deperne, Boulogne-Billancourt (FR)

(73) Assignee: Peters Surgical, Boulogne-Billancourt (FR)

(21) Appl. No.: 18/271,141

(22) PCT Filed: Jan. 7, 2022
(86) PCT No.: PCT/FR2022/050046

§ 371 Date: Jul. 6, 2023

(30) Foreign Application Priority Data
Jan. 7, 2021 (FR) FR2100134

(51) Int. Cl. A61B 17/122

(52) U.S. Cl. CPC A61B 17/122

(57) ABSTRACT

Disclosed is a surgical staple (1) comprising a first jaw (2) and a second jaw (4) designed to pivot relative to each other about a pivot axis (X) in such a way as to clamp biological tissue, in which the first jaw (2) comprises a plurality of teeth (14) suitable for being in contact with the biological tissue clamped by the first jaw (2) and the second jaw (4), the plurality of teeth (14) comprising: a first tooth (14a) having a first surface (28a) that is inclined relative to the pivot axis (X) in a cutting plane parallel to the pivot axis (X) and passing through the first tooth (14a), a second tooth (14b) having a second surface (28b) that is inclined relative to the pivot axis (X), the first surface (28a) and the second surface (28b) being arranged to intersect in a cross-sectional view of the first jaw (2) according to the cutting plane, in which the first tooth (14a) further comprises: a third surface (32a) that is inclined relative to the pivot axis (X), the third surface (32a) being connected to the first surface (28a) and/or to the second surface (28a), and a fourth surface (34a) that is inclined relative to the pivot axis (X), the third surface (32a) and the fourth surface (34a) forming two slopes opposite each other relative to a vertex of the first tooth (14a).

### FIELD OF THE INVENTION

The present invention relates to a surgical staple (also called a surgical “clip” in the literature). In particular, a surgical staple finds application for clamping a biological tissue, typically having a tubular shape, such as a blood vessel.

### PRIOR ART

Known from document WO 2016/205343 is a surgical staple comprising two jaws for clamping a vessel. Each of these jaws comprises a plurality of teeth.

The teeth are formed in two internal surfaces of the jaws that are facing one another. These teeth have the advantage of better clipping the staple to the clamped vessel. However, the teeth proposed in this document do not allow obtaining optimal clipping.

### DISCLOSURE OF THE INVENTION

One object of the invention is to more effectively clip a surgical staple to a biological tissue such as a blood vessel.

To this end a surgical staple is proposed, according to a first aspect, comprising a first jaw and a second jaw designed to pivot relative to one another around a pivot axis so as to clamp a biological tissue, in which the first jaw comprises a plurality of teeth suitable for being in contact with the biological tissue clamped by the first jaw and the second jaw, the plurality of teeth comprising:
a first tooth having a first surface that is inclined relative to the pivot axis in a section plane parallel to the pivot axis and passing through the first tooth,
a second tooth having a second surface that is inclined relative to the pivot axis, the first surface and the second surface being arranged to intersect in a section view of the first jaw in the section plane, in which the first tooth also comprises: a third surface that is inclined relative to the pivot axis, the third surface being connected to the first surface and/or to the second surface, and a fourth surface that is inclined relative to the pivot axis, the third surface and the fourth surface forming two slopes opposite to one another relative to a tip of the first tooth.

The fact that the inclined surfaces of the teeth intersect allows favoring an effective multidirectional clipping of the staple to a biological tissue.

The surgical staple according to the first aspect can comprise the following optional features, taken alone or combined together when that is technically possible.

Preferably, the first surface and the second surface are arranged to intersect in the section view at a point located at mid-width of the first jaw, the width of the jaw being measured parallel to the pivot axis.

Preferably, at least one surface among the first surface and the second surface is flat.

Preferably, the first jaw has:
an internal surface suitable for being in contact with the biological tissue clamped by the first jaw and the second jaw, the plurality of teeth being formed in the internal surface,
two opposite lateral surfaces forming respectively, with the internal surface, a first edge and a second edge opposite to the first edge relative to the internal surface, the first surface extending until the second edge and/or the second surface extending until the first edge.

Preferably, the first jaw has:
an internal surface suitable for being in contact with the biological tissue clamped by the first jaw and the second jaw, the plurality of teeth being formed in the internal surface,
two opposite lateral surfaces forming respectively, with the internal surface, a first edge and a second edge opposite to the first edge relative to the internal surface, the first tooth having a tip that is closer to the first edge than to the second edge and/or the second tooth having a tip closer to the second edge than to the first edge.

Preferably:
the first jaw has an internal surface suitable for being in contact with the biological tissue clamped by the first jaw and the second jaw, the plurality of teeth being formed in the internal surface,
the first jaw has an external surface opposite to the internal surface,
the lateral surfaces comprise respectively two internal lateral portions flaring relative to one another until the first edge and the second edge in a direction running from the external surface toward the internal surface.

Preferably:
one of the internal lateral portions has a tangent, at one point of the first edge, which is inclined by an angle relative to an axis parallel to the pivot axis, the angle being nonzero and strictly less than 90 degrees, perhaps less than or equal to 80 degrees, perhaps less than or equal to 70 degrees, and/or
the other internal lateral portion has a tangent, at one point of the second edge, which is inclined by an angle relative to an axis parallel to the pivot axis, the angle being nonzero and strictly less than 90 degrees, perhaps less than or equal to 80 degrees, perhaps less than or equal to 70 degrees.

Preferably, the plurality of teeth comprises a third tooth, the third surface being inclined relative to the pivot axis so as to be facing the third tooth.

Preferably, the second surface extends between the first tooth and the third tooth.

Preferably, the second jaw comprises a tooth arranged to extend between the first tooth and the third tooth, when the biological tissue is clamped by the first jaw and the second jaw.

Preferably, the first jaw has:
an internal surface suitable for being in contact with the biological tissue clamped by the first jaw and the second jaw, the plurality of teeth being formed in the internal surface,
an external surface opposite to the internal surface,
two lateral surfaces opposite to one another relative to the external surface, the two lateral surfaces comprising respectively two external lateral portions approaching one another until the external surface, in a direction running from the internal surface toward the external surface.

Preferably, the external surface has a convex curved profile in a section plane, and the two external lateral portions have respective rectilinear profiles in the section plane.

Preferably, the two external lateral portions continuously prolong the external surface without forming any roughness.

Moreover, a surgical staple is proposed, according to a second aspect, comprising a first jaw and a second jaw designed to clamp a biological tissue, in which the first jaw has:
an internal surface suitable for being in contact with the biological tissue clamped by the jaws,
an external surface opposite to the internal surface,
two opposite lateral surfaces forming respectively, with the internal surface, two edges, the lateral surfaces comprising respectively two internal lateral portions flaring relative to one another until the edges in a direction running from the external surface toward the internal surface.

The fact that the two internal lateral portions flare relative to one another until the edges allows better clipping the staple to a vessel, compared to a staple comprising two parallel lateral surfaces.

Moreover a surgical staple is proposed, according to a third aspect, comprising a first jaw and a second jaw designed to clamp a biological tissue in which the first jaw has:
an internal surface suitable for being in contact with the biological tissue clamped by the first jaw and the second jaw,
an external surface opposite to the internal surface,
two lateral surfaces opposite to one another relative to the external surface, the two lateral surfaces comprising respectively two external lateral portions approaching one another until the external surface, in a direction running from the internal surface toward the external surface.

Due to the external lateral portions approaching one another until the external surface in a direction running from the internal surface toward the external surface, the proposed staple has fewer tendencies to get jammed in a groove than a staple of which the two lateral surfaces are parallel to one another and connected to an external surface by two edges.

The staples according to the second aspect and according to the third aspect can also comprise all the other features of the staple according to the first aspect which are discussed above.

### DESCRIPTION OF THE FIGURES

Other features, objects and advantages of the invention will be revealed by the description that follows, which is purely illustrative and not limiting, and which must be read with reference to the appended figures, which are the following.

FIG. 1 is a side view of a staple according to one embodiment.

FIG. 2 is a perspective front view of the staple of FIG. 1.

FIG. 3 is a side view of the staple of FIG. 1 and of a staple applicator in an open state.

FIG. 4 is a side view of the staple of FIG. 1 and of a staple applicator in a closed state.

FIG. 5 is another perspective view of the staple of FIG. 1.

FIG. 6 is a partial perspective view of the staple of FIG. 1, centered on an internal surface of a jaw of the staple.

FIG. 7 is a section view in a first section plane of the jaw shown in FIG. 6.

FIG. 8 is a partial perspective view of the staple of FIG. 1, centered on an internal surface of a jaw of the staple.

FIG. 9 is a section view in a second section plane of the jaw shown in FIG. 8.

FIG. 10 is a section view of the staple of FIG. 1, while the staple is in a closed state.

FIG. 11 is a section view of the jaw shown in FIG. 8, in the second section plane.

FIG. 12 is another section view of the jaw shown in FIG. 8, in the second section plane.

In all the figures, similar elements bear identical reference symbols.

### DETAILED DESCRIPTION OF THE INVENTION

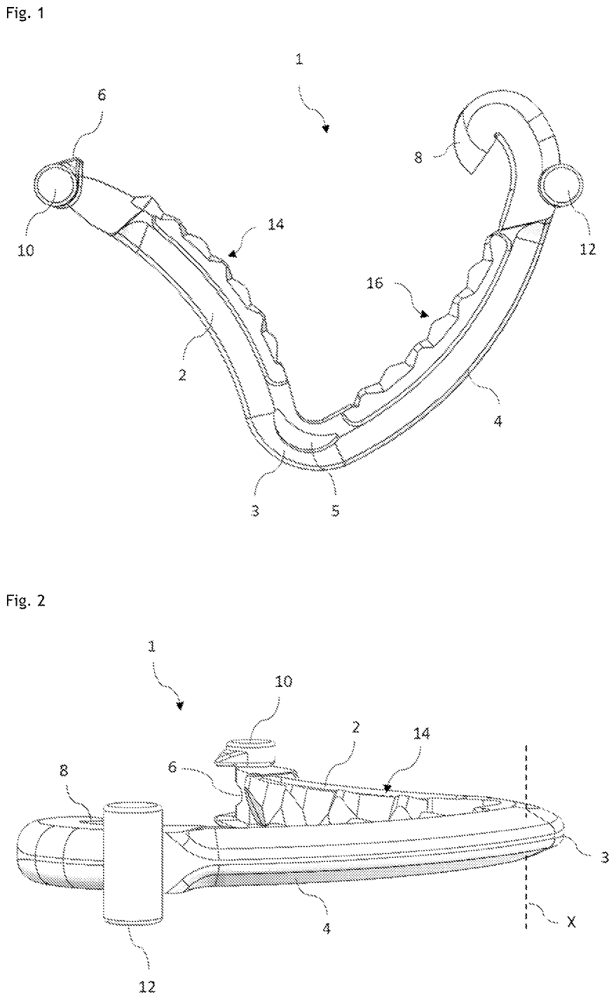

With reference to FIGS. 1 and 2, a surgical staple 1 to clamp a biological tissue, typically having a tubular shape such as a blood vessel, comprises a first jaw 2 and a second jaw 4 able to pivot relative to the first jaw.

The staple 1 comprises a link 3 connecting the first jaw 2 to the second jaw 4. The link 3 forms a hinge in that it allows the second jaw 4 to pivot relative to the first jaw 2 around a pivot axis X, between an open position (visible in FIG. 1) in which the two jaws 2, 4 are separated from one another, and a closed position in which the two jaws 2, 4 are closer to one another so as to be able to clamp a biological tissue.

The staple 1 is a single piece. The link 3 deforms to close and open the staple 1. To favor this deformation, the link 3 has a through recess 5 forming a space between the two arms linking the first jaw 2 to the second jaw 4.

The staple 1 comprises locking means 6, 8 to hold the two jaws 2, 4 in the closed position.

These locking means comprise a first locking element 6 and a second locking element 8 designed to be engaged with the first locking element 6 by elastic interlocking, when the two jaws 2, 4 are brought closer together by pivoting around the pivot axis X.

The first jaw 2 comprises the first locking element 6, and the second jaw 4 comprises the second locking element 8. The first locking element 6 is a male element, and the second locking element 8 is a female element (forming a hook).

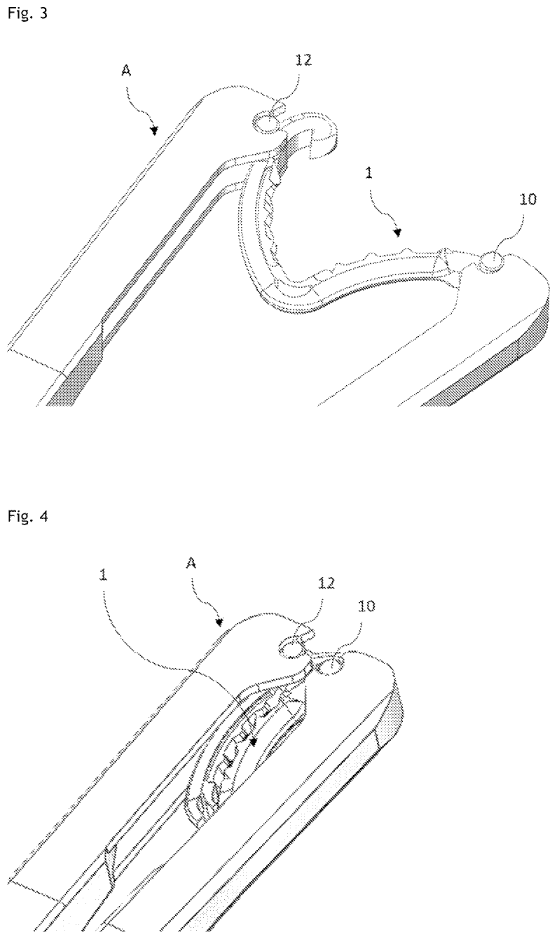

Moreover, the staple 1 comprises two pairs of bosses 10, 12. The pair of bosses 10 is comprised in the first jaw 2, and the other pair of bosses 12 is comprised in the second jaw 4. The two pairs of bosses 10, 12, are arranged to be engaged with two pairs of hooks of an applicator A partially shown in FIGS. 3 and 4, this applicator A having in particular the function of closing the staple 1. The bosses of the pairs 10, 12 have shapes of revolution, so as to be able to turn without binding in the corresponding hooks of the applicator A.

Returning to FIGS. 1 and 2, the first jaw 2 has an elongate shape extending from the link 3 until a free end. The first locking element 6, as well as the pair of bosses 10, are located in proximity to this free end.

The first jaw 2 comprises a first plurality of teeth 14 suitable for being in contact with a biological tissue clamped by the first jaw 2 and the second jaw 4. The first plurality of teeth 14 extends between the first locking element 6 and the link 3.

Similarly, the second jaw 4 has an elongate shape extending from the link 3 until another free end. The second locking element 8 as well as the pair of bosses 12 are located in proximity to this other free end.

The second jaw 4 comprises a second plurality of teeth 16 suitable to be in contact with the biological tissue clamped by the first jaw 2 and the second jaw 4. The second plurality of teeth 16 extends between the second locking element 8 and the link 3.

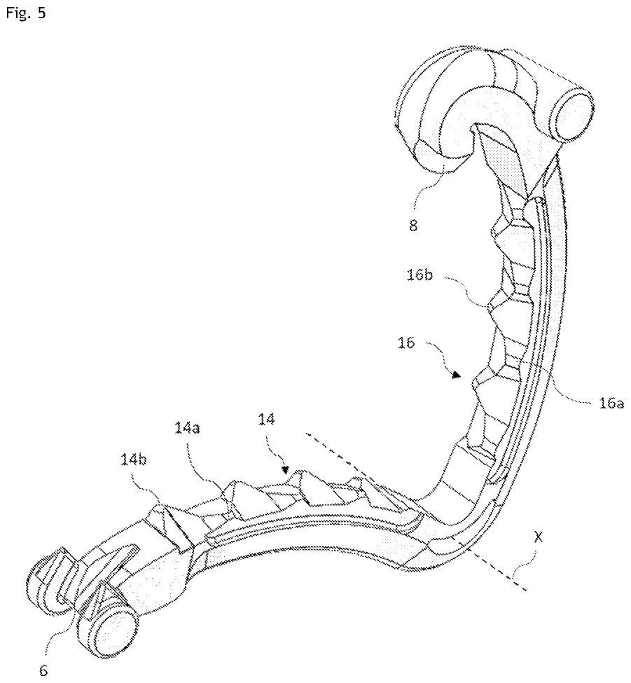

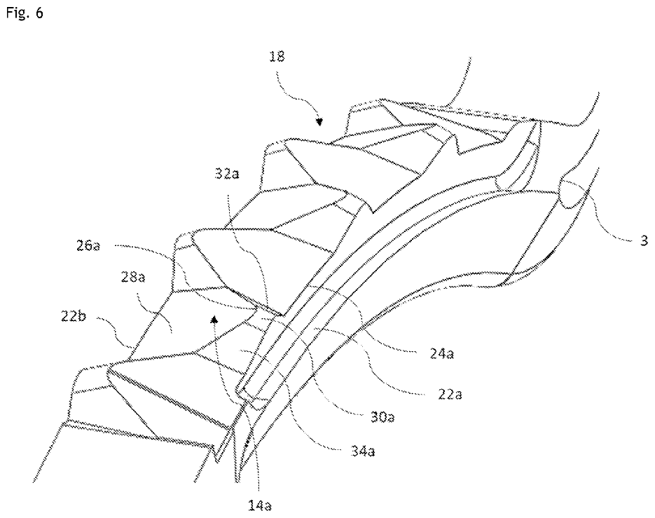

With reference to FIG. 5, the first plurality of teeth 14 comprises two rows of teeth 14a, 14b. Each row of teeth 14a, 14b extends between the link 3 and the locking element 6. The two rows 14a, 14b are alternated between the link 3 and the locking element 6. When passing along the first plurality of teeth 14, starting from the tooth closest to the link 3 and finishing with the tooth closest to the locking element 6, a tooth belonging to the row 14a and a tooth belonging to the other row 14b are alternately encountered.

Similarly, the second plurality of teeth 16 comprises two rows of teeth 16a, 16b. Each row 16a, 16b extends between the link 3 and the locking element 8. The two rows of teeth 16a, 16b are alternated between the link 3 and the locking element 8. When passing along the second plurality of teeth 16 starting from the tooth closest to the link 3 and finishing with the tooth closest to the locking element 8, a tooth belonging to the row 16a and a tooth belonging to the other row 16b are alternately encountered.

The first plurality of teeth 14 and the second plurality of teeth 16 are arranged so as to interlock with one another when the staple 1 is in the closed position. One tooth of one of the jaws 2, 4 is then located between two adjacent teeth of the other jaw, and this applies to any tooth of the plurality of teeth 14 and of the plurality of teeth 16, except the teeth closest to the link 3 and the locking elements 6, 8.

More precisely, when the staple 1 is in the closed position, the rows of teeth 14a and 16a are interlocked with one another, while the rows of teeth 14b and 16b are also interlocked with one another.

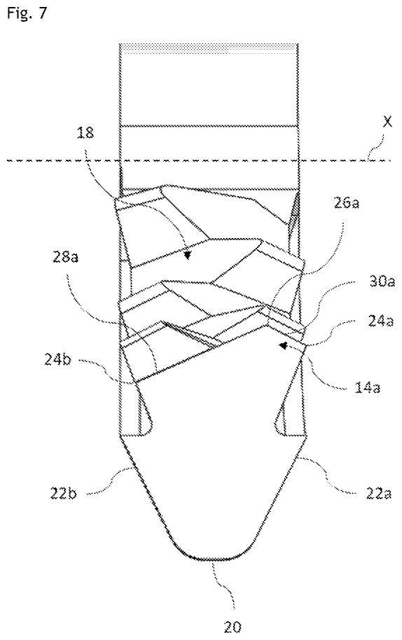

With reference to FIGS. 6 and 7, the first jaw 2 has an internal surface 18, an external surface opposite to the internal surface 18, and two lateral surfaces 22a, 22b opposite to one another and each connecting the internal surface 18 to the external surface 20.

The internal surface 18 is arranged to be facing the second jaw 4 (more precisely, facing the second plurality of teeth 16), when the staple 1 is in the closed position. The internal surface 18 is suitable for being in contact with a biological tissue clamped by the first jaw 2 and the second jaw 4.

The first plurality of teeth 14 is formed in the internal surface 18.

The internal surface 18 is connected to the lateral surface 22a by an edge 24a, and moreover connected to the other lateral surface 22b by another edge 24b. The two edges 24a, 24b form two opposite sides of the internal surface 18.

In the present text, the term edge will implicitly designate a live edge, i.e. a protruding edge of any shape. Thus, an edge within the meaning of the present text is not limited to a rectilinear shape, but can have other shapes, particularly curved shapes.

Noted previously is the fact that the first plurality of teeth 14 comprises two alternating rows of teeth 14a, 14b. The row of teeth 14a is closer to the edge 24a than to the other edge 24b. In other words, the row of teeth 14a is closer to the lateral surface 22a than to the other lateral surface 22b. By contrast, the row of teeth 14b is closer to the edge 24b than to the other edge 24a. In other words the row of teeth 14b is closer to the lateral surface 22b than to the other lateral surface 22a.

Description of a Tooth 14a

The shape of any one of the teeth 14a will now be described with reference to FIGS. 6 and 7, knowing that this description is applicable to each of the other teeth of the same row.

The tooth 14a has a tip 26a closer to the edge 24a than to the other edge 24b. In other words, the tip 26a is closer to the lateral surface 22a than to the other lateral surface 22b.

The tooth 14a is formed by the plurality of portions 28a, 30a, 32a, 34a of the internal surface 18, which join at the tip 26a.

The surface portion 28a, called the primary side surface 28a, extends from the top 26a of the tooth until the edge 24b which is farthest away.

The primary side surface 28a is inclined with respect to the pivot axis X. Preferably, the inclination angle of the main side surface 28a relative to the pivot axis X, measured in a first section plane parallel to the pivot axis X, is comprised between 5 and 45 degrees, perhaps between 10 and 30 degrees, for example 20 degrees. The first section plane considered (that of FIG. 7) passes through the tip 26a and crosses the internal surface 18, the external surface 20 as well as the lateral surfaces 22a, 22b of the first jaw 2.

The primary side surface 28a is for example flat. The aforementioned angle of inclination is then constant. In this case, the primary side surface 28a defines, with the lateral surface 22b, a rectilinear segment of the edge 24b, visible in FIG. 6.

The primary side surface 28a is moreover arranged so as to be facing the tip of a tooth 16a of the second jaw 4, when the staple 1 is closed.

The surface portion 30a, called the secondary side surface 30a, extends from the tip 26a of the tooth 14a to the edge 24a closest to the tip 26a. The primary side surface 28a and the rear secondary side surface 30a form two slopes opposite to one another relative to the tip 26a of the tooth 14a.

The secondary side surface 30a is inclined with relation to the pivot axis X. Preferably, the angle of inclination of the secondary side surface 30a relative to the pivot axis X in the section plane discussed above (that of FIG. 7) is comprised between 5 and 45 degrees, perhaps between 10 and 30 degrees, for example 20 degrees. The respective angles of inclination of the side surfaces 28a, 30a relative to the pivot axis X can in particular be equal in the first section plane.

The secondary side surface 30a has for example a convex curve. In this case, the secondary side surface defines a curved portion of the edge 24a, visible in FIG. 6.

The side surfaces 28a, 30a are connected to one another by a short edge. The tip 26a of the tooth 14a is a point of this curved edge.

The surface portion 32a, called the rear surface 32a, is oriented toward the link 3. More precisely, the rear surface 32a is inclined relative to the pivot axis X, so as to be facing a tooth belonging to the same row as the tooth 14a considered, and which is located closer to the link 3.

The rear surface 32a is connected to the primary side surface 28a at a rear edge. The rear surface 32a is for example flat. In this case, the rear edge is a segment.

The surface portion 34a, called the front surface 34a, is oriented toward the locking element 6. The front surface 34a and the rear surface 32a form two slopes opposite to one another relative to the tip 26a of the tooth 14a. The front surface 34a is inclined relative to the pivot axis X so as to be facing another tooth belonging to the same row as the tooth 14a considered, and which is located closer to the free end of the first jaw 2.

The front surface 34a is connected to the primary side surface 28a at a front edge. The front surface 34a is flat. In this case, the front edge is a segment.

The front surface 34a and the rear surface 32a can be symmetrical relative to a plane passing through the tip 26a of the tooth (the plane of FIG. 7).

The rear surface 32a, the front surface 34a and the secondary side surface 30a can form together a convex surface free of roughness. In this case the secondary side surface 30a forms a fillet making a continuous junction between the rear surface 32a and the front surface 34a.

Description of a Tooth 14b

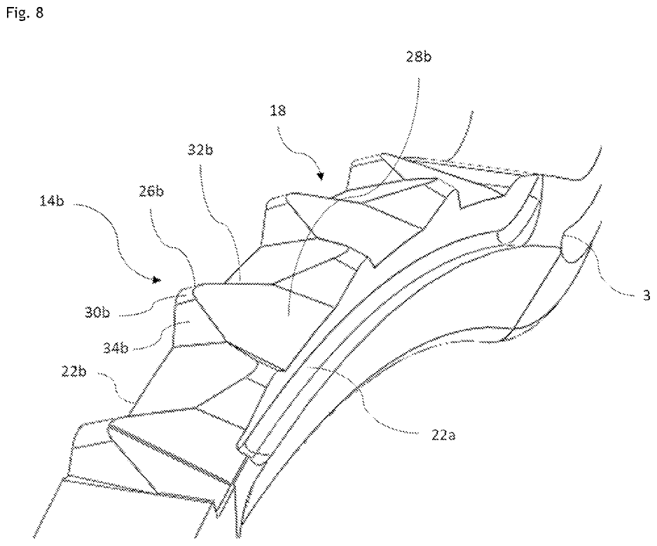

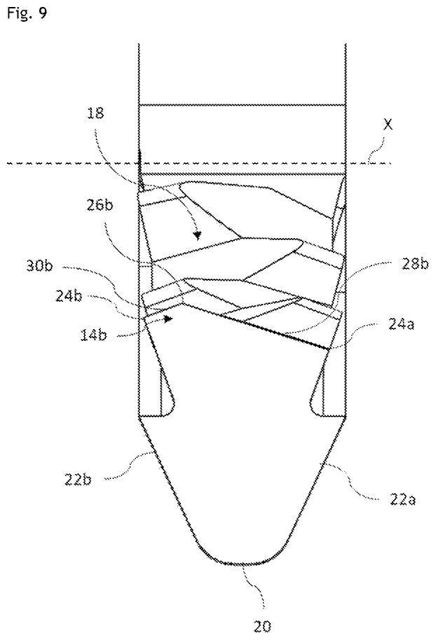

The shape of any one of the teeth 14b will now be described with reference to FIGS. 8 and 9, knowing that this description is applicable to all of the other teeth of the same row.

The tooth 14b has a tip 26b closer to the edge 24b than to the other edge 24a. In other words, the tip 26b is closer to the lateral surface 22b than to the other lateral surface 22a.

The tooth 14b is formed by a plurality of portions 28b, 30b, 32b, 34b of the internal surface 18, which join at the tip 26b.

The surface portion 28b, called the primary side surface 28b, extends from the tip 26b of the tooth until the edge 24a, which is the one farthest away.

The primary side surface 28b is inclined relative to the pivot axis X. Preferably, the angle of inclination of the main side surface 28b relative to the pivot axis X, measured in a second section plane parallel to the pivot axis X, is comprised between 5 and 45 degrees, perhaps between 10 and 30 degrees, for example 20 degrees. The second section plane considered is the plane of FIG. 9, passing through the tip 26b, and crossing the internal surface 18, the external surface 20 as well as the lateral surfaces 22a, 22b of the first jaw 2.

The primary side surface 28b is for example flat. The aforementioned angle of inclination is then constant. In this case, the primary side surface 28b defines, with the lateral surface 22a, a rectilinear segment of the edge 24a.

Moreover, the primary side surface 28b is arranged so as to be facing the tip of a tooth 16b of the second jaw 4 when the staple 1 is closed.

The surface portion 30b, called the secondary side surface 30b, extends from the tip 26b of the tooth 14b until the edge 24a closest to the tip 26b. The primary side surface 28b and the secondary side surface 30b form two slopes opposite to one another relative to the tip 26b of the tooth 14b.

The secondary side surface 30b is inclined relative to the pivot axis X. Preferably, the angle of inclination of the secondary side surface 30b relative to the pivot axis X in the second section plane is comprised between 5 and 45 degrees, perhaps between 10 and 30 degrees, for example degrees. The respective angles of inclination of the side surfaces 28b, 30b relative to the pivot axis X can in particular be equal in the second section plane.

The secondary side surface 30b has for example a convex curve. In this case, the secondary side surface defines a curved portion of the edge 24b.

The side surfaces 28b, 30b are connected to one another at a curved edge. The top 26b of the tooth 14b is a point of this curved edge.

The surface portion 32b, called the rear surface 32b, is oriented toward the link 3. More precisely, the rear surface 32b is inclined relative to the pivoting axis X, so as to be facing a tooth belonging to the same row and the tooth 14b considered, and which is located closer to the link 3.

The rear surface 32b is connected to the primary side surface 28b by a rear edge. The rear surface 32b is for example flat. In this case the rear edge is a segment.

The surface portion 34b, called the front surface 34b, is oriented toward the locking element 6. The front surface 34b and the rear surface 32b form two slopes opposite to one another relative to the tip 26b of the tooth 14b. The front surface 34b is inclined relative to the pivot axis X, so as to be facing another tooth belonging to the same row as the tooth 14b considered, and which is located closer to the free end of the first jaw 2.

The front surface 34b is connected to the primary side surface 28b at a front edge. The front surface 34b is flat. In this case the front edge is a segment.

The front surface 34b and the rear surface 32b can be symmetrical relative to a plane passing through the tip 26b of the tooth (the plane of FIG. 9).

The rear surface 32b, the front surface 34b and the secondary side surface 30b can form together a convex surface free of roughness. In this case, the secondary side surface 30b forms a fillet making a continuous junction between the rear surface 32b and the front surface 34b.

Relative Positioning Between the Teeth 14a, 14b

Each tooth 14a is adjacent to a tooth 14b, and vice versa.

The rear surface 32a of a tooth 14a is connected to the primary side surface 28b of a tooth 14b that is adjacent to it, so as to form a concavity.

Moreover, the front surface 34a of the tooth 14a is connected to the primary side surface 28b of another tooth 14b which is adjacent to it, so as to form a concavity.

The tip 26a and the secondary side surface 30a of the tooth 14a are localized between the respective primary side surfaces 28b of these two adjacent teeth 14b.

The primary side surface 28a of the tooth 14a is connected to the front surface 34b of one of the teeth 14b which are adjacent to it, while forming a concavity. The primary side surface 28a of the tooth 14a is also connected to the rear surface 32b of the other tooth 14b which is adjacent to it, while forming a concavity. The primary side surface 28a thus extends between the two adjacent teeth 14b, more precisely between the respective tips 26b of these two adjacent teeth 14b.

Reciprocally, the rear surface 32b of a tooth 14b is connected to the primary side surface 28a of a tooth 14a which is adjacent to it, while forming a concavity.

Moreover, the front surface 34b of the tooth 14b is connected to the primary side surface 28a of another tooth 14a which is adjacent to it, while forming a concavity.

The tip 26b and the secondary side surface 30b of the tooth 14b are localized between the respective primary side surfaces 28a of these two adjacent teeth 14a.

The primary side surface 28b of the tooth 14b is connected to the front surface 34a of one of the teeth 14a which are adjacent to it, while forming a concavity. The primary side surface 28b of the tooth 14b is also connected to the rear surface 32a of the other tooth 14a which is adjacent to it, while forming a concavity. The primary side surface 28b thus extends between the two adjacent teeth 14a, more precisely between the respective tips 26a of these two adjacent teeth 14a.

Moreover, two teeth 14a, 14b adjacent to one another have respective primary side surfaces 28a, 28b which intersect in a section view of the first jaw 2 in a section plane parallel to the pivot axis X. This is verified in particular in the first section plane (that of FIG. 7) and the second section plane (that of FIG. 9) previously discussed. Such an arrangement favors a multidirectional clipping of the teeth 14 that is more effective on the biological tissue clamped by the staple 1.

The intersection point of the surfaces 28a, 28b in this view is located at mid-width of the first jaw 2, this width of the jaw being measured in a direction parallel to the pivot axis X.

The intersection discussed above is applicable to any pair of adjacent teeth 14a, 14b of the first jaw 2, and any pair of teeth 16a, 16b of the second jaw.

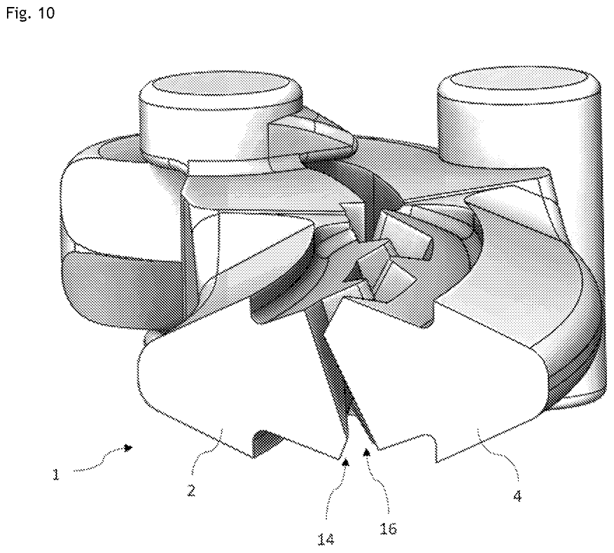

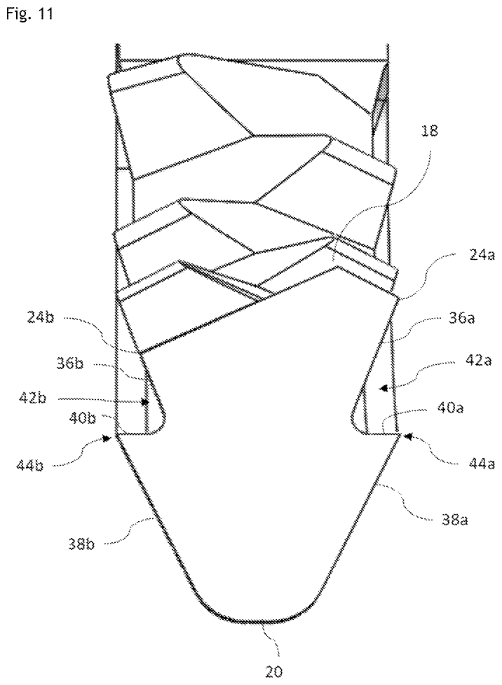

With reference to FIG. 10, a tooth 14a and a tooth 16a facing one another delimit between them a space forming a chicane, when the staple is closed. This is also true for a pair of teeth 14b, 16b facing one another. The tissue clamped by the staple 1 extends into this space.

Description of the Lateral Surfaces 22a, 22b and of the External Surface 20

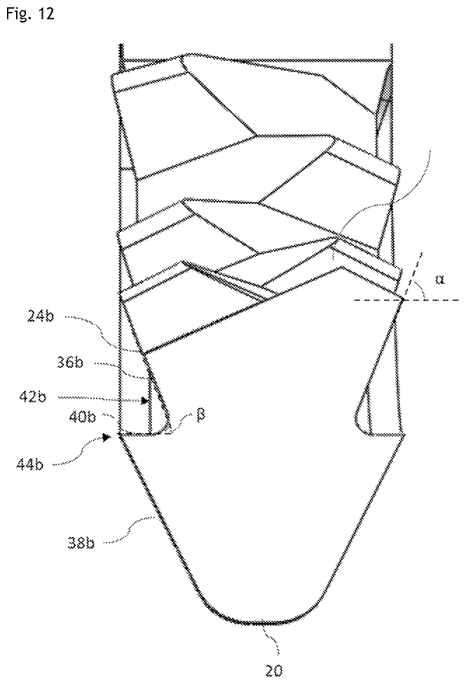

With reference to FIG. 11, the external surface 20, opposite to the internal surface 18 previously discussed, has the purpose of being located facing the bottom of a groove of the applicator A.

The external surface 20 is a convex curved surface.

The lateral surface 22a of the first jaw 2 comprises a surface portion 36a which is connected to the internal surface 18 at the edge 24a.

The surface 36a has a tangent, at a point of the edge 24a, which is inclined by an angle α relative to an axis parallel to the pivoting axis X. The angle α is nonzero and strictly less than 90 degrees. This thus allows making the edge 24a more protruding. Preferably, the angle α is less than or equal to 80 degrees, perhaps less than or equal to 70 degrees. This angle α is encountered in particular in the first section plane of FIGS. 7 and 11, and/or in the second section plane of FIG. 9).

The surface 36a can have, in a section plane, a rectilinear profile in the section plane considered. Moreover, the lateral surface 22a comprises a surface portion 38a which is connected to the external surface 20, continuously for example, i.e. without forming an edge with the external surface 20.

Moreover, the lateral surface 22a comprises an intermediate surface portion 40a. The intermediate surface portion 40a forms, with the surface portion 36a, a longitudinal groove 42a extending between the link 3 and the free end of the first jaw 2.

Moreover, the intermediate surface portion 40a forms, with the surface portion 38a, a longitudinal rib 44a extending between the link 3 and the free end of the first jaw 2. The intermediate surface portion 40a is connected to the surface portion 38a by a longitudinal edge forming, on the one hand, an edge line of the longitudinal rib 44a and moreover an edge of the longitudinal groove 42a.

Similarly, the lateral surface 22b comprises a surface portion 36b which is connected to the internal surface 18 at the edge 24b.

The surface 36b has a tangent, at a point of the edge 24b, which is inclined by an angle θ relative to an axis parallel to the pivot axis X. The angle θ is nonzero and strictly less than 90 degrees. This thus allows making the edge 24b more protruding. Preferably, the angle θ is less than or equal to 80 degrees, perhaps less than or equal to 70 degrees. This angle θ is encountered in particular in the first section plane of FIGS. 7 and 11, and/or in the second section plane of FIG. 9.

The surface 36b can have, in a section plane, a rectilinear profile in the section plane considered.

Moreover, the lateral surface 22b comprises a surface portion 38b which is connected to the external surface 20, continuously for example, i.e. without forming an edge with the external surface 20.

Moreover, the lateral surface 22b comprises an intermediate surface portion 40b. The intermediate surface portion 40b forms, with the surface portion 36b, a longitudinal groove 42b extending between the link 3 and the free end of the first jaw 2.

Moreover, the intermediate surface portion 40b forms, with the surface portion 38b, a longitudinal rib 44b extending between the link 3 and the free end of the first jaw 2. The intermediate surface portion 40b is connected to the surface portion 38b by a longitudinal edge forming, on the one hand, an edge line of the longitudinal rib 44b, and moreover an edge of the longitudinal groove 42b.

The surfaces 36a, 36b flare relative to one another until the edges 24a, 24b in a direction running from the external surface 20 toward the internal surface 18. In other words, the width of the first jaw measured in a direction parallel to the pivot axis X increases, when passing along the surfaces 36a, 36b toward the edges 22a, 22b. A flaring of this type allows the formation of more protruding angles at the edges 24a, 24b. These edges 24a, 24b form two sides of the internal surface 18 having the purpose of being in contact with a biological tissue clamped between the jaws 2, 4 of the staple. Consequently, the edges 22a, 22b can clip the biological tissue. Making them more protruding allows improving this clipping.

Typically, the internal surface 18 and the lateral surface 22a form at the edge 24a an angle less than 100 degrees in a section plane of the jaw passing through the internal surface 18, the external surface 20 and the two lateral surfaces 22a, 22b. Similarly, the internal surface 18 and the lateral surface 22b form at the edge 24b an angle less than 100 degrees in a section plane of the jaw passing through the internal surface 18, the external surface 20 and the lateral surfaces 22a, 22b. Such angles are found in particular in the first section plane and in the second section plane. It should be noted that these angles would be further reduced in the embodiments (not illustrated) in which the surface 18 is flat and parallel to the pivot axis X (in other words, in the absence of teeth 14).

Moreover, the surfaces 38a, 38b approach one another until the external surface 20, in a direction running from the internal surface toward the external surface 20. This approach begins at the ribs 44a, 44b. This approach confers upon the jaw a tapered profile on the outside.

With a tapered profile of this type, the first jaw has fewer tendencies to get jammed in the applicator, compared to a jaw with a rectangular profile.

Moreover, it will be noted that the presence of the surfaces 40a, 40b allows obtaining both the flaring in proximity to the edges 24a, 24b and the tapered profile at the external surface, without however significantly increasing the width of the jaw 2.

All the features of the first jaw 2 described above are also found in the second jaw 4. In particular:
All the features of a tooth 14a described above are also found in a tooth 16a;
All the features of a tooth 14b described above are also found in a tooth 16b;
The second jaw 4 has an external surface comprising the same features as the external surface 20 of the first jaw 2;
The second jaw 4 has two lateral surfaces comprising the same features as the lateral surfaces 22a, 22b of the first jaw 2.

1. A surgical staple comprising a first jaw and a second jaw designed to pivot relative to one another around a pivot axis so as to clamp a biological tissue, wherein the first jaw comprises a plurality of teeth suitable for being in contact with the clamped biological tissue clamped by the first jaw and the second jaw, the plurality of teeth comprising:
a first tooth having a first surface that is inclined relative to the pivot axis in a section plane parallel to the pivot axis and passing through the first tooth, and
a second tooth having a second surface that is inclined relative to the pivot axis, the first surface and the second surface being arranged to intersect in a section view of the first jaw in the section plane, wherein the first tooth further comprises:
a third surface that is inclined relative to the pivot axis, the third surface being connected to at least one of the first surface and the second surface, and
a fourth surface that is inclined relative to the pivot axis, the third surface and the fourth surface forming two slopes opposite to one another relative to a tip of the first tooth.

2. The staple according to claim 1, wherein the first surface and the second surface are arranged to intersect in the section view at a point located at mid-width of the first jaw, a width of the jaw being measured parallel to the pivot axis.

3. The staple according to claim 1, wherein at least one of the first surface and the second surface is flat.

4. The staple according to claim 1, wherein the first jaw has:
an internal surface suitable for being in contact with the biological tissue clamped by the first jaw and the second jaw, the plurality of teeth being formed in the internal surface, and
two opposite lateral surfaces forming respectively, with the internal surface, a first edge and a second edge opposite to the first edge relative to the internal surface, the first surface extending until the second edge and/or the second surface extending until the first edge.

5. The staple according to claim 1, wherein the first jaw has:
an internal surface suitable for being in contact with the biological tissue clamped by the first jaw and the second jaw, the plurality of teeth being formed in the internal surface, and
two opposite lateral surfaces forming respectively, with the internal surface, a first edge and a second edge opposite to the first edge relative to the internal surface, the first tooth a tip that is closer to the first edge than to the second edge and/or the second tooth having a tip closer to the second edge than to the first edge.

6. The staple according to claim 4, wherein:
the first jaw has an internal surface suitable for being in contact with the biological tissue clamped by the first jaw and the second jaw, the plurality of teeth being formed in the internal surface,
the first jaw has an external surface opposite to the internal surface, and
the lateral surfaces comprise respectively two internal lateral portions flaring relative to one another until the first edge and the second edge in a direction running from the external surface toward the internal surface.

7. The staple according to claim 6, wherein:
one of the internal lateral portions has a tangent, at one point of the first edge, which is inclined by an angle relative to an axis parallel to the pivot axis, the angle being nonzero and strictly less than 90 degrees, and/or
the other internal lateral portion has a tangent, at a point of the second edge, which is inclined by an angle relative to an axis parallel to the pivot axis, the angle being nonzero and strictly less than 90 degrees.

8. The staple according to claim 1, wherein the plurality of teeth comprises a third tooth, the third surface being inclined relative to the pivot axis so as to be facing the third tooth.

9. The staple according to claim 8, wherein the second surface extends between the first tooth and the third tooth.

10. The staple according to claim 8, wherein the second jaw comprises a tooth arranged to extend between the first tooth and the third tooth, when the biological tissue is clamped by the first jaw and the second jaw.

11. The staple according to claim 1, wherein the first jaw has:
an internal surface suitable to be in contact with the biological tissue clamped by the first jaw and the second jaw, the plurality of teeth being formed in the internal surface,
an external surface opposite to the internal surface, and
two lateral surfaces opposite to one another relative to the external surface, the two lateral surfaces comprising respectively two external lateral portions approaching one another until the external surface, in a direction running from the internal surface toward the external surface.

12. The staple according to claim 11, wherein the external surface has a convex curved profile in the section plane, and the two external lateral portions have respective rectilinear profiles in the section plane.

13. The staple according to claim 11, wherein
the two external lateral portions continuously prolong the external surface without forming any roughness.
